(19) United States
(12) Patent Application Publication
(10) Pub. No.: US 2009/0124870 A1
(43) Pub. Date: May 14, 2009

(54) PATIENT MONITORING SYSTEM FOR THE REAL-TIME DETECTION OF EPILEPTIC SEIZURES

(75) Inventors: Johannes Bernardus Albertus Maria Arends, Waalre (NL); Paulus Antonius Maria Griep, Heeze (NL); In Yu Tan, Deurne (NL)

Correspondence Address: PEACOCK MYERS, P.C., 201 THIRD STREET, N.W., SUITE 1340, ALBUQUERQUE, NM, 87102, US

(73) Assignee: Hobo Heeze B.V., Heeze (NL)

(21) Appl. No.: 12/277,552

(22) Filed: Nov. 25, 2008

Related U.S. Application Data
(63) Continuation-in-part of application No. PCT/NL2007/050264, filed on Jun. 5, 2007.

(30) Foreign Application Priority Data
Jun. 7, 2006 (NL) 1031958

(51) Int. Cl. A61B 5/00

(52) U.S. Cl. 600/301

(57) ABSTRACT

A patient monitoring system for the real-time detection of epileptic seizures suffered by a user of the monitoring system. The system comprises control means for receiving measuring signals and, conditional upon the measuring signals, generating an alarm signal, at least one hart rate sensor for measuring the heart rate of the user and, subject to the measurement, generating a heart rate signal, and at least one muscular tension sensor for measuring the change and the intensity of the contraction of at least one muscle of the user and, subject to the measurement, generating a muscle contraction signal. The control means are designed for, subject to both the heart signal and the muscle contraction signal, generating an alarm signal when the user suffers an epileptic seizure.

### CROSS-REFERENCE TO RELATED APPLICATIONS

This application is a continuation-in-part application of international Patent Application Serial No. PCT/NL2007/050264, entitled “patient Monitoring System for the Real-Time Detection of Epileptic Seizures”, to Hobo Heeze B. V., filed on Jun. 5, 2007, and the specification and claims thereof are incorporated herein by reference.

This application claims priority to and the benefit of the filing of Netherlands Patent Application Serial No. 1031958, entitled “Patient Monitoring System for the Real-Time Detection of Epileptic Seizures”, filed on Jun. 7, 2006, and the specification and claims thereof are incorporated herein by reference.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### COPYRIGHTED MATERIAL

Not Applicable.

### BACKGROUND OF THE INVENTION

1. Field of the Invention (Technical Field)

The present invention relates to a patient monitoring system for the real-time detection of epileptic seizures suffered by a user of a monitoring system, which comprises control means for receiving measuring signals and for generating an alarm signal conditional upon the measuring signals.

2. Description of Related Art

Such patient monitoring systems are known, and are used for monitoring people suffering from epilepsy, for example when these people are not staying in a hospital or other medical facility where they may be monitored permanently.

Epilepsy is the third most prevalent neurological condition: approximately 1 in 150 people suffers from it, and in the Netherlands several hundred patients die each year from the consequences of epilepsy. Epilepsy manifests itself in many forms: auras, absences, myoclonics, (small changes in muscle tension), clonic seizures (jerking), tonic seizures (convulsion), etc.

Approximately 70% of epileptic patients are able to almost completely suppress the seizures by means of medication or surgery. However, this is not possible for the remainder of patients. These patients' functioning is restricted by the seizures, and they may injure themselves and others as a result of a seizure. A severe, prolonged seizure may in itself even lead to a life-threatening situation, requiring medical intervention (status epilepticus). Another occurrence is SUDEP (“Sudden Unexplained Death in Epilepsy”), killing approximately one epileptic patient in three thousand each year.

A particular group of patients is comprised of people suffering from very severe epilepsy, as this is also often coupled with mental and physical handicaps. This means they are dependent on carers and often live in a specialized environment.

It goes without saying that sometimes, especially with the more severe seizures, assistance may be needed. A patient monitoring system, warning social care workers by means of alarms and at the same time informing them of the person's whereabouts, is of real assistance in such a case. In an intramural situation (within a health care institution) this could improve the quality of the care, while reducing the work load of the carers.

For the group of patients suffering from a “milder” form of epilepsy, who have sporadic seizures, such a system acts as a safety net and provides reassurance for them and those surrounding them. Because they will feel less tied to the ‘safe’ home situation, this group will have more freedom of movement. The knowledge that seizures will be detected, enabling social care workers/care givers to react, lowers the threshold for them to admit to go out more often and thus contributes to their quality of life.

Prior art systems currently used for this purpose comprise, for example, systems using video and/or fairly simple sound measurement optionally supplemented with, for example, moisture sensors placed in the bed, vibration sensors, or oxygen saturation measurements on the patient. These may be remotely monitored systems and they register, for example, urine loss, jolting of the bed as a result of the person's muscle contractions during the seizure or, in the case of an oxygen saturation measurement (Sp02 measurement) the oxygen content in the blood, which is indicative of the activity of the patient's breathing. If, for example in the latter case, the patient stops breathing, the oxygen content in the blood drops and the system will emit an alarm signal.

In accordance with the prior art, numerous monitoring systems are known that register heart rate changes and based on these, emit an alarm signal. Such systems may also be used for monitoring a patient and for diagnosing an epileptic seizure.

A drawback of all the above—mentioned monitoring systems is that none of the systems provides a truly reliable alarm signal. For example, if a moisture sensor is placed in the bed, and the person loses urine during an epileptic seizure, the urine does not necessarily come in contact with the moisture sensor and the loss of urine goes entirely unnoticed, without a signal from the sensor. It is also possible that the person in bed perspires excessively, causing the moisture sensor to emit a signal without any epileptic seizure actually occurring. Thus, apart from generating many false alarms, such a sensor also lets a number of epileptic seizures go undetected.

Similar drawbacks are also attached to placing vibration sensors, for example, under a mattress. During an epileptic seizure the bed may possibly vibrate as a result of changes in the patients muscular tension and this can be measured with the aid of vibration sensor in the bed. However, vibrations may have many other causes, so that in a number of cases, a system based on vibration sensors will likewise generate false alarms. It is also possible that the changes in the patient's muscular tension are not strong enough, or that the patient's position in bed is such that the vibrations caused by changes in the patient's muscular tension are inadequately transmitted to the mattress and to the vibration sensors. In that case, the epileptic seizures will go completely unnoticed. Another drawback is that in many cases the epileptic seizure is not accompanied by the patient experiencing periodic tensional changes in certain muscles, so that it is quite likely that an epileptic seizure goes entirely unnoticed.

An alarm system based on oxygen saturation measurements may in many cases also generate a false alarm. It is known that the oxygen content in the blood is not equally high at all times and may fluctuate due to the breathing pattern and differences in the manner and measure of the patient's breathing. Especially during sleep it is possible that people for some moments breathe only lightly, taking in relatively little oxygen. On the basis of an oxygen saturation measurement, this could easily generate a false alarm.

Such an argument also applies to heart rate monitoring systems. These systems are not specifically designed for detecting epileptic seizures, they detect heart rate changes in general. It is known that the occurrence of heart rate changes is not uncommon, while not necessarily involving an epileptic seizure. The use of monitoring systems that only monitor the person's heart rate will therefore in many cases also generate false alarms.

In all the above cases there is the risk of false alarms or of failing to detect epileptic seizures altogether. It has been shown that when the system is insufficiently reliable, generating many false alarms, the alertness of the attending personnel also declines. This may result in an alarm signal failing to procure quite the appropriate reaction, so that such a system affords the person to be monitored a false security.

### BRIEF SUMMARY OF THE INVENTION

It is an object of the present invention to provide a patient monitoring system for the detection of epileptic seizures and the emission of an alarm signal, facilitating the reliable detection of epileptic seizures.

### DESCRIPTION OF THE INVENTION

The present invention achieves this objective by providing a patient monitoring system for the real-time detection of epileptic seizures suffered by a user of the monitoring system, comprising control means for receiving measuring signals and, subject to the measuring signals, generating an alarm signal, at least one hart rate sensor for measuring the heart rate of the user and, subject to the measurement, generating a heart rate signal, and at least one muscular tension sensor for measuring the change and the intensity of the contraction of at least one muscle of the user and, subject to the measurement, generating a muscle contraction signal, wherein the control means are designed for, subject to both the heart signal and the muscle contraction signal, generating an alarm signal when the user suffers an epileptic seizure.

The invention is based on the understanding that epileptic seizures can be electronically identified in a reliable manner by combining heart rate measurements with measurements relating to the change and intensity of contractions of one or several of the user's muscles. This greatly reduces the risk of generating a false alarm or the risk of the epileptic seizure going completely unnoticed. Moreover such measurements may be executed fairly easily by using relatively simple sensors. It is, therefore, not necessary to fit the user of the patient monitoring system with a great number of sensors and the inconvenience to the user of the patient monitoring system is kept to a minimum, which enhances user-friendliness.

The data stream provided by the above-mentioned sensors is small, requiring only simple processing means in the patient monitoring system for generating an alarm signal. The patient monitoring system according to the present invention will therefore consume only little electric energy, wireless embodiments are no problem. Remarkably, the inventors have come to the insight that in order to obtain a reliable signal for patient monitoring, relatively little measuring data is required. In this respect the invention distinguishes itself from, for example, diagnostic methods for which a physician is preferably provided with as much data as possible, even if it bears only the slightest relevance.

In accordance with one embodiment of the invention, the control means are designed for analyzing the muscle contraction signal. Studies have shown that the change in contraction intensity of some muscles during an epileptic seizure affords considerable insight with regard to the type of seizure suffered by a person.

An epileptic seizure may also be recognized by how the heart rate changes during the seizure. In accordance with one embodiment, the control means of the patient monitoring system is therefore designed for analyzing the heart rate signal. With the aid of the analysis of the heart signal and/or the analysis of the muscle contraction signal, the control means may be equipped to typify an epileptic seizure, wherein the alarm signal provided by the control means may be indicative of the type of epileptic seizure.

The detection of patterns in the heart rate is based on the fact that a large number of patients show a rise in heart rate followed by a fall. This occurs according to a patient specific, but characteristic pattern that may be described as follows: a linear rise, followed by a plateau and finally an exponential fall, possible with an “undershoot”. A heart beat decline in the reverse pattern also occurs in a limited number of patients.

The detection of epileptic seizures by means of analyzing the change in muscle contractions (3D-ACM) may occur in combination with a number of steps: first a distinction may be made between day and night situations. Subsequently, contraction may be distinguished from no contraction, and the former periods may be scanned for epilepty form activity. Finally, the detected seizures are subdivided into types: myoclonic, tonic or (tonic-) clonic. This occurs by classification on the basis of features derived from models considered to be specific for these types of seizures.

Based on the analysis of the measured signals (heart rate and how the intensity of the muscular tension changes) it is possible to reliably establish the type of epileptic seizure. Thus the alarm signal that is indicative of the type of epileptic seizure can be used to provide medical support geared to the type of epileptic seizure the patient is suffering. Also, the severity of the epileptic seizure can be assessed directly.

In accordance with a further embodiment, the patient monitoring system further comprises at least one additional sensor for providing measuring signals indicative of the body functions of the user, for providing additional information relating to the epileptic seizure.

Although the person skilled in the art will appreciate that increasing the number of sensors will also increase the data stream, and the increasing data stream may influence the design of the patient monitoring system as well as its simplicity, the advantages of additional information may in some cases outweigh the negative effect this has on the design of the patient monitoring system. Sensors to be considered are in particular sensors selected from a group comprising: respiratory sensors, neurological sensors for measuring neurological activity such as electro-encephalographic sensors, temperature sensors, sound sensors, impedance sensors for measuring the impedance of the skin, blood pressure sensors, sensors for determining the moisture content in the body, oxygen saturation sensors, light sensors, external moisture sensors, etc.

In accordance with various embodiments, signals may be transmitted wirelessly between the sensors and the control means, in particular between additional sensors and the control means and between the muscular tension sensor and heart rate sensor and the control means. To this end the patient monitoring system may be equipped with traditional means for the wireless transmission of signals, for example, based on Bluetooth, zigbee, and other electromagnetic transmitter-receiver means. The person skilled in the art will appreciate that, when the control means are incorporated in a housing carried on the body of the user, the distance which the signals to be transmitted have to bridge will usually not exceed approximately 1 meter. It is also possible for the sensors to be attached to the body of the user, while the control means and the housing are located, for example, in the home of the user. In this case the distance to be bridged will generally be kept to some tens of meters.

In accordance with a further embodiment, the patient monitoring system may be equipped for transmitting the alarm signals to be generated to a receiver. This may, for example, be a centre observing the patient monitoring systems of several users and, if necessary, taking appropriate medical action in the event of an alarm. The signals may simply be transmitted to the centre, for example, via a telecommunication network. The alarm signals may be transmitted, for example, via internet, or by means of a regular telephone network. It is also possible to set up the patient monitoring system such that it is suitable for transmitting the signals via a mobile telecommunication network, for example, based on UMTS, GSM, and other known mobile telecommunication protocols.

However, if the central unit of the patient monitoring system is comprised of a housing in which the control means are incorporated and which is permanently positioned in the home of a user, it is simple to provide a regular connection to the internet or, with the aid of a traditional modem provide contact with the central unit via a telephone network. Any other ways for the transmission of alarm signals to a receiver, for example to a central monitoring service, will be obvious to the person skilled in the art.

In accordance with a further embodiment, the patient monitoring system comprises positioning means for providing position signals indicating the system's location. If the system is carried on the user's body, such a system may operate, for example, based on GPS, Galileo or, for example triangulation of the signal to be transmitted by the patient monitoring system.

If the patient monitoring system is embodied with such positioning means, the user can be located as soon as an alarm occurs. If immediate medical action is required, the centre can, for example, inform an ambulance service of the location of the user.

In accordance with a further embodiment of the invention, the patient monitoring system comprises recording means for storing signals transmitted to the control means. These recordings means may be comprised of magnetic recording carriers, but in practice it will be more convenient to use memory chips able to store data from the sensors for some length of time. This makes it possible to analyze the signals received from the various sensors some time after the occurrence of an epileptic seizure. It will be obvious to the person skilled in the art that this is advantageous for the treatment of patients suffering from epilepsy.

### BRIEF DESCRIPTION OF THE SEVERAL VIEWS OF THE DRAWINGS

The invention described above will be further explained by way of non-limiting examples of such systems, with reference to the appended drawings, in which:

FIG. 1 schematically shows a patient monitoring system in accordance with the invention, with which alarm signals can be transmitted to a central monitoring service.

FIG. 2 schematically shows an apparatus in accordance with the invention;

FIG. 3 shows the typical curve of a heart rate change that may occur during an epileptic seizure; and

FIG. 4 shows the typical curve of muscular contraction changes during an epileptic seizure, and the logical stimuli responsible for this.

### DETAILED DESCRIPTION OF THE INVENTION

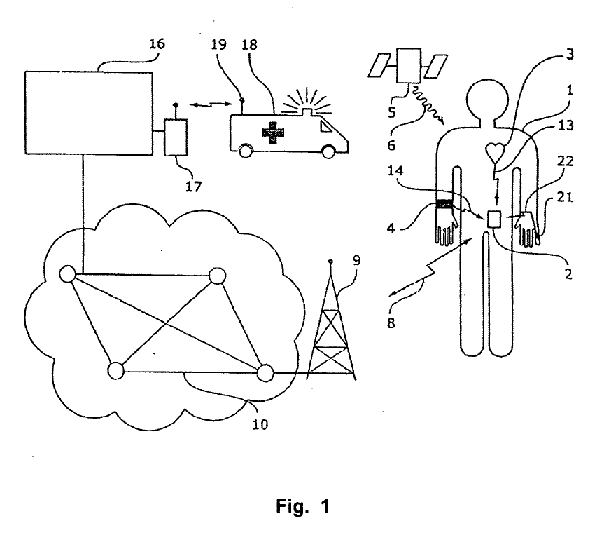

FIG. 1 schematically shows a patient monitoring system in accordance with the present invention, carried by a patient 1. The user 1 carries a central unit 2, which consists of a housing comprising a processing unit (not shown), alarm unit (not shown) and transmission means (not shown). The person skilled in the art will appreciate that units and means of this kind may be embodied differently. On the body of the user 1, for example in the vicinity of the heart, a heart rate sensor 3 is provided and in addition, for example near the end of an extremity, a muscular tension sensor, 4 which is capable of measuring the changes and intensity of muscular tension in the extremity. In the embodiment shown in FIG. 1, the signals from heart rate sensor 3 and muscular tension sensor 4 are transmitted wirelessly to central unit 2, as schematically indicated by arrows 13 and 14.

When the user 1 suffers an epileptic seizure, this will be accompanied by, for example, a change in heart rate, and depending on the type of epileptic seizure, muscular tension changes. If the sensor 3 measures the heart rate of the patient continuously, the processing unit (not shown) in the central processing unit in the central unit 2 will detect this heart rate change. The muscle contractions continuously measured with the aid of a muscle tension sensor 4 are also analyzed by the processing unit (not shown) in the central unit 2, and the muscular tension changes typical of epileptic seizures can be recognized by the processing unit. Based on the signals received from both the heart rate sensor 3 and the muscular tension sensor 4, the processing unit will establish the probability of an epileptic seizure occurring, and based on these data may emit an alarm signal. With the aid of transmission means, this alarm signal can be transmitted to, for example, a receiving device 9 in a wireless telecommunication network and, with the aid of the telecommunication network 10, communicated to the central monitoring service 16. The wireless transmission of the alarm signal from the central unit to the receiving device 9 is schematically represented with double arrow 8. The double arrow 4 also indicates that, in some embodiments of the system, the central monitoring service may possibly challenge the central unit 2 for additional information relating to the epileptic seizure. To this end, signal exchange has to take place between the central monitoring service 16 and the central unit order to allow two-way signal transmission between receiver device 9 and central unit 2.

Based on the alarm signal received, the central monitoring service 16 is able to take appropriate action. In the event of a severe epileptic seizure with considerable risks of complications, the central monitoring service may, for example, elect to provide immediate medical assistance on site. To this end the central monitoring service may, for example with the aid of transmission means 7, transmit a signal to an ambulance service, schematically represented as ambulance 18. The ambulance 18 may, for example, be equipped with a receiver unit 19, receiving the signal from the central monitoring service 16 provided by transmission unit 17, in order to proceed to the location where the user 1 of the patient monitoring system finds himself during the epileptic seizure.

In order to determine the exact location of the user 1 of the patient monitoring system, the central unit 2 may be equipped with, for example, a GPS system or another locating system. In the case of a GPS system or Galileo system, the central unit 2 receives signals 6 emitted by a plurality of satellites 5 (FIG. 1 schematically shows only one satellite, but the person skilled in the art will be acquainted with GPS positioning systems).

The patient monitoring system may also comprise additional sensors, such as an additional Sp0₂ sensor 21 which is able, for example, to measure the oxygen content in the blood of the user 1. The signal provided by sensor 21 may also be transmitted wirelessly to the central unit 2, as schematically represented by arrow 22.

In accordance with the invention it is possible to obtain a highly reliable signal for determining epileptic seizures by combining a heart rate measurement and a measurement of the intensity of muscular tension changes. The additional sensors, such as additional sensor 21, are optional and their purpose is to provide additional information that might be necessary or desirable for medical assistance. The information from these sensors may also be used to improve the quality of the alarm signal. The patient monitoring system according to the invention is already operative when a heart rate sensor 3 and muscle tension sensor 4 are employed. The alarm signal during an epileptic seizure is generated primarily on the basis of signals received from sensors 3 and 4, and it is possible that none of the data from sensor 21 plays a part in the analysis performed by the processing unit and central unit 2, but is only communicated to the central monitoring service. Particular attention is drawn to the fact that the use and the function of measuring signals from the various sensors (3, 4, 21) connected with the user consists, on the one hand, of providing signals for the reliable detection of an epileptic seizure for alarm purposes and, on the other hand, of providing signals for obtaining insight into the medical situation. With respect to the first case, the emission of signals for generating the alarm signal, a limited analysis of the most essential signals is required, whereas with respect to the second case, providing information for medical purposes, the measured signals from sensors (21) only need to be communicated to the central monitoring service (16) without requiring further analysis. This utilization of signals from different sensor allows for a simple design of the patient monitoring system, as well as an efficient management of resources. The fewer functionalities are built in, the fewer elements are involved in the design and the smaller the data stream. The result will be that the energy consumption will also be kept low so that it is possible to work with smaller batteries. Thus the design may be kept small, causing the user 1 of such a patient monitoring system as little inconvenience as possible.

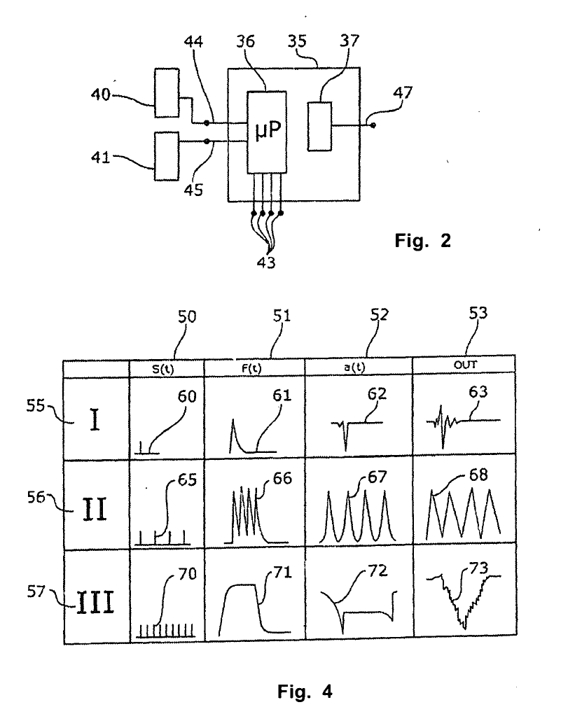

The system in accordance with the invention is again shown schematically in FIG. 2. The central unit 35 comprises in any case a micro processor 36. The central unit 35 shown in FIG. 2 also comprises a transmission unit 37 able to communicate the alarm signal provided via output 47 to, for example, a telecommunication network or other suitable means. A person skilled in the art will appreciate that the output 47 may optionally also be connected with, for example, a loudspeaker or other means for an audible reproduction of the alarm signal. The manner in which the alarm signal is communicated to the external world is chosen subject to the situation in which the patient monitoring system is employed by the user.

On the micro processor 36, two sensors 40 and 41 are connected via inputs 44 and 45. Sensor 40 is a heart rate sensor and sensor 41 is a muscular tension sensor, both similar to the sensors shown in the embodiment described in FIG. 1. The person skilled in the art will appreciate that any arbitrary number of sensors may be used for providing the signals with which the patient monitoring system operates. In each of the embodiments shown in FIGS. 1 and 2, one muscular tension sensor and one heart rate sensor is used. If desired, two, three, four, five or more muscular tension sensor and/or heart rate sensors may be used, for example to provide a reliable signal. In its simplest form, the patient monitoring system according to the invention is already operable with one single heart rate sensor and one single muscular contraction sensor. A plurality of inputs 43 affords the possibility of connecting additional sensors to the patient monitoring system, for example, to provide additional data concerning the epileptic seizure. Although the embodiments illustrated in FIG. 2 only show four inputs/outputs 43, this number may optionally be increased or reduced to any chosen number of inputs and outputs for additional sensors. The inputs and outputs 43 may optionally also even be omitted completely, since the connection of additional sensors is optional.

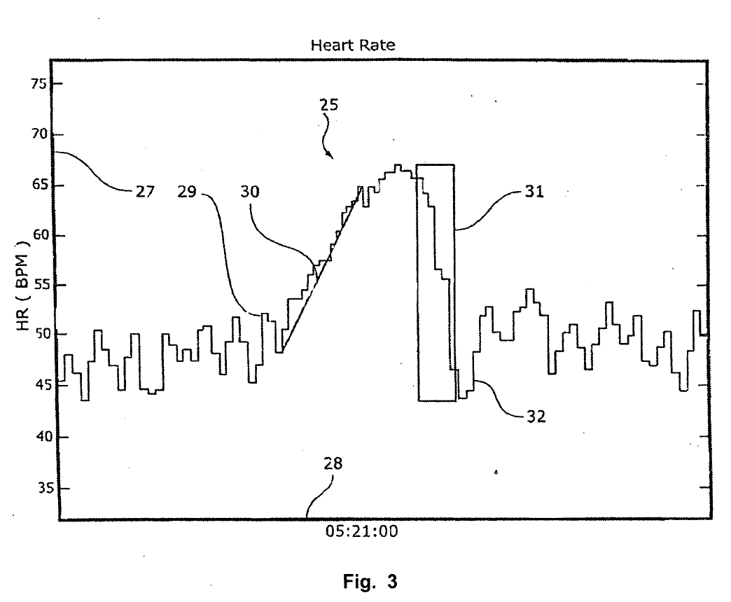

FIG. 3 shows an example of how a heart rate changes during an epileptic seizure. This change is schematically represented by reference numeral 25. The vertical axis 27 in the graph of FIG. 3 relates to the heart rate, while the horizontal axis 28 relates to time. Thus the signal represented by the jagged line 29 shows the change of the heart rate overtime. When an epileptic seizure occurs, the heart rate will first exhibit a linear rise, indicated by the linear fit 30, after which it will change to a strong exponential fall, represented by the area in box 31. The strong exponential fall shown in box 31 may lead to an ‘undershoot’ 32, with the heart rate falling to a local minimum. Such a heart rate change is characteristic during the occurrence of an epileptic seizure and may be used for its detection.

FIG. 4 further shows the curve of the intensity of muscular tension changes (F(t)) for various types of epileptic seizures. The types of epileptic seizures are indicated by I, II and III. I in row 55 of the table corresponds to a myoclonic seizure. II in row 56 of the table corresponds to a clonic seizure. III in row 57 corresponds to an epileptic seizure of the tonic type. Combinations of these types of epileptic seizures, such as tonic-clonic, can also be identified by means of a system according to the invention. In the table of FIG. 4 only a few types of seizures will be described, although the person skilled in the art will appreciate that, in addition to the types of epileptic seizures shown in FIG. 4, there are other types of epileptic seizures that can also be identified by means of the present methods.

In the first column 50 of the table, the neurological-stimuli responsible for the occurrence of the epileptic seizure are schematically represented by function S(t). In the second column 51 of the table, the curve of the intensity of muscular tension changes F(t) is represented schematically. The output signal measured on a muscular tension sensor is modulated, and the simulated output signal a(t) is represented schematically in column 52. Column 53 in FIG. 4 shows the measured output signal of the muscular tension sensor.

For a myoclonic seizure (Table Type I) a single neurological stimulus 60 may be responsible. Such a stimulus produces a single muscular tension change 61 in the muscles of the user. The user may be making an unexpected movement that may be compared to the occurrence of a sudden discharge in the muscles causing a jolt through the body of a healthy person during the first stage of sleep. Patients suffering from a form of epilepsy involving myoclonic seizures may experience such seizures often and all through the day. The simulated output signal from a muscular tension sensor is shown, carrying reference numeral 62 and in the last column 53 of the table in FIG. 4 a real signal is shown, measured by a muscular tension sensor. When the myoclonic seizure-specific ‘spike’ occurs, the micro processor b transmits an alarm signal to the central monitoring service.

The signals that are measured during the occurrence of a clonic seizure (Table Type II) may be described as follows. The neurological stimuli S(t) are represented as a series of short pulses, carrying reference numeral 65. These stimuli produce muscular tension changes in the patient's muscles that are indicated by reference numeral 66. The modulated output signal and the real output signal from a patient suffering such a seizure are indicated by reference numeral 67 and 68.

In row 57 of the table in FIG. 4, the curve of a tonic seizure (Table Type III) is shown. Here also, the neurological stimuli 70 consist of a series of pulses following each other in such quick succession, however, that the curve of the muscular tension changes 71 during such a seizure will no longer be pulsed as is the case with a clonic seizure (indicated by reference numeral 66), but instead, the muscle will temporarily become rigid as indicated by reference numeral 71.

The modulated output signal is again shown in column 52 carrying reference numeral 72. In reality, the output signal will in such a case resemble that shown in column 53, carrying reference numeral 73.

Combining the heart rate measurements with measurements of the changing intensity of the muscle contractions reduces the risk of overlooking an epileptic seizure to a minimum, while simultaneously forestalling a large number of false alarms. The moment an alarm is generated, the central monitoring service can take appropriate medical action. In response to an alarm, the central monitoring service may, for example, elect to telephone the user of the patient monitoring system and provide personal advice. To this end the patient monitoring system according to the invention may optionally be equipped with means enabling the central monitoring service to contact the user. Contact may, for example, be appropriate during the occurrence of one or several light myoclonic seizures. In the event of the user suffering a more severe epileptic seizure, for example, a prolonged tonic-clonic seizure, the central monitoring service may elect to provide immediate medical assistance on site, and to call an ambulance service. The fact that the system generates far fewer false alarms, greatly reduces the risk of an ambulance service being dispatched to a user of a patient monitoring system unnecessarily. The person skilled in the art will appreciate that this is a direct advantage of the invention.

In the foregoing the invention has been described with reference to non-limiting specific embodiments. However, the protective scope of the invention is limited by the appended claims only.

What is claimed is:

1. A patient monitoring system for the real-time detection of epileptic seizures suffered by a user of the monitoring system, comprising control means for receiving measuring signals and, conditional upon the measuring signals, generating an alarm signal, at least one hart rate sensor for measuring the heart rate of the user and, subject to the measurement, generating a heart rate signal, and at least one muscular tension sensor for measuring the change and the intensity of the contraction of at least one muscle of the user and, subject to the measurement, generating a muscle contraction signal, wherein the control means are designed for, subject to both the heart signal and the muscle contraction signal, generating an alarm signal when the user suffers an epileptic seizure.

2. A patient monitoring system according to claim 1, wherein the control means are designed for analyzing the muscle contraction signal.

3. A patient monitoring system according to claim 1, wherein the control means are designed for analyzing the heart rate signal.

4. A patient monitoring system according to claim 2, wherein the control means are designed, on the basis of the analysis, to typify the epileptic seizure, and wherein the alarm signal is indicative of the type of epileptic seizure.

5. A patient monitoring system according claim 1, further comprising means for the wireless transmission to the control means of at least one of the signals selected from the group comprising the heart rate signal, the muscle contraction signals.

6. A patient monitoring system according claim 1, further comprising at least one additional sensor for providing measuring signals indicative of the body functions of the user, for providing additional information relating to the epileptic seizure.

7. A patient monitoring system according to claim 6, wherein the at least one additional sensor is selected from the group consisting of respiratory sensors, neurological sensors for measuring neurological activity, electroencephalographic sensors, temperature sensors, sound sensors, impedance sensors for measuring the impedance of the skin, blood pressure sensors, sensors for determining the moisture content in the body, oxygen saturation sensors, light sensors, and external moisture sensors.

8. A patient monitoring system according to claim 6, further comprising means for wireless transmission to the control means of the measuring signals from at least one of the additional sensors.

9. A patient monitoring system according to claim 8, wherein the control means further comprises transmission means for transmitting the alarm signal to a receiver.

10. A patient monitoring system according to claim 9, wherein the control means are further designed for, with the aid of the transmission means, transmitting at least one of the measuring signals from at least one sensor selected from the group consisting of the heart rate sensor, the muscle contraction sensor and the additional sensors.

11. A patient monitoring system according to claim 9, wherein the transmission means are designed for wireless transmission of the alarm signal and/or the at least one measuring signal.

12. A patient monitoring system according to claim 9, wherein the transmission means are designed for the transmission of the alarm signal and/or the at least one measuring signal by means of a telecommunication network.

13. A patient monitoring system according to claim 12, wherein the transmission means are designed for transmission of the alarm signal by means of an internet connection.

14. A patient monitoring system according to claim 9, further comprising positioning means for providing position signals giving an indication of the system's location.

15. A patient monitoring system according to claim 9, further comprising recording means for storing 40 signals transmitted to the control means.
